(19) United States
(12) Patent Application Publication
(10) Pub. No.: US 2026/0020817 A1
(43) Pub. Date: Jan. 22, 2026

(54) DYNAMIC AND CUSTOMIZED ADJUSTMENT OF SAFETY CONSTRAINTS FOR A MEDICAMENT DELIVERY SYSTEM

(71) Applicant: INSULET CORPORATION, Acton, MA (US)

(72) Inventors: Mert SEVIL, Manchester, NH (US); Yibin ZHENG, Hartland, WI (US); William WHITELEY, Knoxville, TN (US); Saeed SALAVATI, Sugar Land, TX (US)

(21) Appl. No.: 19/249,496

(22) Filed: Jun. 25, 2025

Related U.S. Application Data
(60) Provisional application No. 63/671,954, filed on Jul. 16, 2024.

(51) Int. Cl. A61B 5/00; G16H 20/10

(52) U.S. Cl. CPC A61B 5/4839; A61B 5/7282; G16H 20/10

(57) ABSTRACT

Exemplary embodiments may customize the safety constraints of a medicament delivery system for a user. The customization may occur on an ongoing basis so as to dynamically adapt to the changing medicament needs of the user and thus provide better therapeutic results for the user. The safety constraints may be adjusted at periodic intervals, such as daily, hourly, or even per operational cycle of the control system of the medicament delivery system. The adjustments may be based on recent performance of the control system in providing desired therapeutic results. The safety constraint(s) that are adjusted may be those that were invoked more than the other safety constraints in a recent interval.

### CROSS-REFERENCE TO RELATED APPLICATIONS

This application claims priority to and the benefit of U.S. Provisional Application No. 63/671,954, filed Jul. 16, 2024, the entirety of which is incorporated herein by reference.

### BACKGROUND

Most conventional Automated Insulin Delivery (AID) systems employ safety constraints to mitigate risks, especially severe hypoglycemia. Unfortunately, such safety constraints may hinder performance of the AID systems. Tight safety constraints may limit the amount of insulin that may be delivered to a user and may limit the rate of adaptability of the control system (i.e., how quickly the control system may respond in adjusting insulin doses). As a result, tight safety constraints may lead to significant hyperglycemia. Conversely, relaxed safety constraints may result in frequent and severe hypoglycemic events due to overshoot of a response or delivery of an excess amount of insulin.

Conventionally, the safety constraints are based on advisory data from norms derived from the general population. This conventional approach is limited as a result. Some users will differ from the general population norms. Some users are more insulin sensitive than the norms and thus the safety constraints for the general population norm tend to enhance their risk of hypoglycemia. On the other hand, some users are less sensitive to insulin than the norm, so the safety constraints for the general population norms tend to enhance the risk of hyperglycemia for such users.

### SUMMARY

In accordance with a first inventive facet, a medicament delivery system includes a non-transitory processor-readable storage media that stores programming instructions and a processor configured for executing the programming instructions. Executing the programming instructions causes the processor to determine a frequency with which each of a plurality of safety constraints is encountered over a period from data for the period and to determine a number of hypoglycemic events and/or a number of hyperglycemic events were experienced by a user over the period. Where the number of hypoglycemic events is determined, based on one or more criteria, a determination may be made of whether the period should be categorized as a hypoglycemic period, and where it is determined that the period should be categorized as a hypoglycemic period, the selected one of the safety constraints that was encountered the most in the period may be tightened. Where the number of hyperglycemic events is determined, based on one or more criteria, a determination may be made of whether the period should be categorized as a hyperglycemic period, and where it is determined that the period should be categorized as a hyperglycemic period, the selected one of the safety constraints that was encountered the most in the period may be relaxed.

The executing of the programming instructions by the processor may cause the processor to, where it is determined that the period should not be categorized as a hypoglycemic period or a hyperglycemic period, determine that the period is a euglycemic period, and may cause the processor to not relax and not tighten the safety constraints. The one or more criteria used to determine that the period should be categorized as a hypoglycemic period may include whether the determined number of hypoglycemic events for the period exceeds a threshold and whether the user experienced a glucose level in the period that was below a floor value. The plurality of the safety constraints may include a one-time constraint that places a maximum on an amount of medicament that can be delivered per a delivery, an integral constraint that places a maximum on an amount of medicament that can be delivered over multiple deliveries, a zero cost medicament constraint that specifies a penalization of a medicament cost term in a cost function for determining basal medicament doses, or an insulin on board constraint. A hyperglycemic event may be when a glucose level of the user exceeds a hyperglycemic threshold, and/or a medicament delivery dosage exceeded a threshold. A hypoglycemic event may be when a glucose level of the user is below a hypoglycemic threshold, and/or a medicament delivery was suspended a threshold number of times.

In accordance with another inventive facet, a medicament delivery system may include a non-transitory processor-readable storage media storing programming instructions and a processor configured for executing the programming instructions. The executing of the programming instructions may cause the processor to analyze recent glucose levels of a user over an interval to determine whether glucose level control was satisfactory. Where glucose level control is determined to be unsatisfactory, at least one of safety constraints imposed by a control system of the medicament delivery system may be adjusted. The at least one safety constraint that may be adjusted may restrict dosing by the medicament delivery system to reduce a risk of hypoglycemia to the user and the adjusting may modify the restriction to make it more or less restrictive based on the prior glucose level control. Where glucose level control is determined to be satisfactory, the safety constraints may not be adjusted. The programming instruction may be stored in a non-transitory processor-readable storage medium.

The analyzing of the recent glucose levels of the user may include determining a number of hypoglycemic events that occurred over the interval. The analyzing of the recent glucose levels of the user may include determining whether any of the recent glucose levels of the user over the interval were below a severe hypoglycemia glucose level threshold. The analyzing of the recent glucose levels of the user may include determining a number of hyperglycemic events that occurred over the interval. The adjusting may adjust a single one of the safety constraints or may adjust multiple ones of the safety constraints. The programming instructions when executed by the processor may further cause the processor to determine a frequency in which respective ones of the safety constraints were invoked to restrict dosing by the control system during the interval. The adjusting may adjust a most frequently invoked safety constraint of the safety constraints during the interval or may adjust multiple ones of more frequently invoked safety constraints of the safety constraints during the interval. The adjusting, for example, may adjust one of the safety constraints that limits dosing to a maximum for a single medicament delivery or that limits dosing to a maximum over a period.

### BRIEF DESCRIPTION OF THE DRAWINGS

FIG. 1 depicts an illustrative medicament delivery system of exemplary embodiments.

FIG. 2 depicts a flowchart of illustrative steps that may be performed in exemplary embodiments as part of a control loop for the medicament delivery system.

FIG. 3A depicts illustrative safety constraints for the medicament delivery system of exemplary embodiments.

FIG. 3B depicts illustrative lower and upper bounds for the safety constraints of FIG. 3A in exemplary embodiments.

FIG. 4 depicts a flowchart of illustrative steps that may be performed in exemplary embodiments as part of periodic processing for the safety constraints.

FIG. 5 depicts a flowchart of illustrative steps that may be performed in exemplary embodiments as part of the hypoglycemic pathway.

FIG. 6 depicts a flowchart of illustrative steps that may be performed in exemplary embodiments to tighten a most limiting constraint (MLC).

FIG. 7 depicts a flowchart of illustrative steps that may be performed in exemplary embodiments as part of the hyperglycemic pathway.

FIG. 8 depicts a flowchart of illustrative steps that may be performed in exemplary embodiments to loosen a most limiting constraint (MLC).

### DETAILED DESCRIPTION

Exemplary embodiments may customize the safety constraints of a medicament delivery system for a user. The customization may occur on an ongoing basis so as to dynamically adapt to the changing medicament needs of the user and thus provide better therapeutic results for the user. The safety constraints may be adjusted at periodic intervals, such as daily, hourly, or even per operational cycle of the control system of the medicament delivery system. The adjustments may be based on recent performance of the control system in providing desired therapeutic results. For example, where the medicament delivery system delivers a medicament for managing glucose levels of the user, the desired results are glucose levels of the user remaining in an acceptable range, such as within a certain range relative to a target glucose level. In the exemplary embodiments, the recent glucose level history of the user may indicate that the glucose levels of the user were too high, too low, or acceptable. The control system of the medicament delivery system may adjust one or more safety constraints to attempt to improve the glucose level control. More generally, the control system may look at recent analyte levels of the user and adjust one or more of the safety constraints based on the analyte level control. Examples of analyte levels include, but are not limited to, glucose levels, ketone levels, heart rate values, blood pressure readings, blood chemistry analyte levels, oxygen saturation levels, perceived pain levels, electrocardiogram values or patterns, electroencephalogram values or patterns, and/or the like.

The control system may decide which safety constraint or constraints to change based on the recent activity of the control system and recent analyte levels. For instance, the control system may determine which of the safety constraints was invoked the most in recent history to constrain medicament delivery by the medicament delivery system. The safety constraint that was invoked the most may be the one that is chosen to be adjusted in some exemplary embodiments. In other exemplary embodiments, multiple safety constraints may be adjusted concurrently. For instance, both the most invoked safety constraint and the second most invoked safety constraint may be adjusted. In some exemplary embodiments, all of the safety constraints may be concurrently adjusted. In other instances, such as when the analyte level control reflects the desired results (e.g., euglycemia or analyte levels within a predefined range, such as 100-150 mg/dL), no adjustments to the safety constraint may be made.

It should be appreciated that the adjustments may tighten the safety constraints so that the levels of medicament that are permitted to be delivered to the user are decreased and/or the rate of adaptation to changing analyte levels is slowed. Conversely, the adjustments may relax the safety constraints so that higher levels of medicament are permitted to be delivered to the user and/or the rate of adaptation to changing analyte levels is increased.

A constraint is a rule or condition that restricts what can be done. For purposes of the exemplary embodiments, a constraint limits certain an action that, but for the constraint, may be taken by the medicament delivery system. A safety constraint is one that limits or restricts to provide safety to the user of the medicament delivery system. For example, a safety constraint may prohibit an action that may lead to hypoglycemia or hyperglycemia for the user or that raises the risk of hypoglycemia or hyperglycemia for the user.

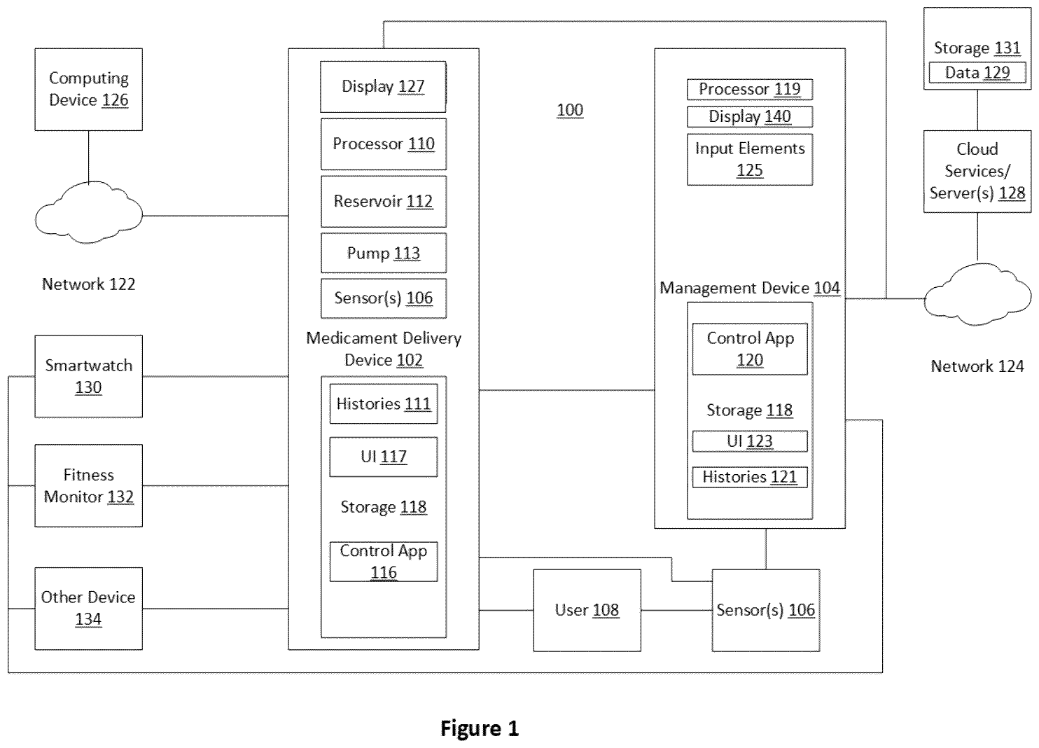

FIG. 1 depicts a block diagram of an illustrative medicament delivery system 100 that is suitable for delivering a medicament to a user 108 in accordance with the exemplary embodiments. The medicament delivery system 100 may include a medicament delivery device 102. The medicament delivery device 102 may be a wearable device that is worn on the body of the user 108 or carried by the user. The medicament delivery device 102 may be directly coupled to the user 108 (e.g., directly attached to a body part and/or skin of the user 108 via an adhesive or the like) with no tubes and an infusion location directly under the medicament delivery device 102, or carried by the user 108 (e.g., on a belt or in a pocket) with the medicament delivery device 102 connected to an infusion site where the medicament is injected using a needle and/or cannula. A surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102 (i.e., to provide the control system). The control application 116 may be a single program, multiple programs, modules, libraries or the like. The processor 110 also may execute computer programming instructions stored in the storage 114 for a user interface (UI) 117 that may include one or more display screens shown on display 127. The display 127 may display information to the user 108 and, in some instances, may receive input from the user 108, such as when the display 127 is a touchscreen.

The control application 116 may control delivery of the medicament to the user 108 per a control approach like that described herein. The control application may use a glucose prediction model as described below for predicting future glucose levels of the user 108. The storage 114 may hold histories 111 for a user, such as a history of basal medicament deliveries, a history of bolus medicament deliveries, and/or other histories, such as a meal event history, exercise event history, glucose level history, other analyte level history, and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include a tray or cradle and/or one or more housings for housing its various components including a pump 113, a power source (not shown), and a reservoir 112 for storing medicament for delivery to the user 108. In some embodiments, a structure, such as part of the housing, may be provided for holding a vial or other source of medicament rather than including a reservoir 112. A fluid path to the user 108 may be provided, and the medicament delivery device 102 may expel the medicament from the reservoir 112 or other medicament source to deliver the medicament to the user 108 using the pump 113 via the fluid path. The fluid path may, for example, include tubing coupling the medicament delivery device 102 to the user 108 (e.g., tubing coupling a cannula to the reservoir 112), and may include a conduit to a separate infusion site. The medicament delivery device 102 may have operational cycles, such as every 5 minutes, in which basal doses of medicament are calculated and delivered as needed, such as each cycle.

There may be one or more communications links with one or more devices physically separated from the medicament delivery device 102 including, for example, a management device 104 of the user 108 and/or a caregiver of the user 108, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN), a cellular network, a Wi-Fi network, a near field communication network, or a combination thereof. A computing device 126 may be interfaced with the network 122, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions, physical attributes, or analyte levels of the user 108. The sensor(s) 106 may be physically separate from the medicament delivery device 102 or may be an integrated component thereof. The sensor(s) 106 may include, for example, glucose monitors, such as continuous glucose monitors (CGM's) and/or non-invasive glucose monitors. The sensor(s) 106 may include ketone sensors, other analyte sensors, heart rate monitors, breathing rate monitors, motion sensors, temperature sensors, perspiration sensors, blood pressure sensors, alcohol sensors, or the like. Some sensors 106 may also detect characteristics of components of the medicament delivery device 102. For instance, the sensors 106 in the medicament delivery device may include voltage sensors, current sensors, temperature sensors and the like.

The medicament delivery system 100 may or may not also include a management device 104. In some embodiments, no management device is needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensor(s) 106. The management device 104 may be any portable electronic device including, for example, a dedicated device, a smartphone, a smartwatch, or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. Storage 118 may also be operable to store historical information such as medicament delivery information, analyte level information, user input information, output information, or other historical information. The control application 120 may be responsible for controlling the medicament delivery device 102, such as by controlling the automated medicament delivery (AMD) (or, for example, automated insulin delivery (AID)) of medicament to the user 108. The storage 118 may store the control application 120, histories 121 like those described above for the medicament delivery device 102, and other data and/or programs.

A display 140, such as a touchscreen, may be provided for displaying information. The display 140 may display user interface (UI) 123. The display 140 also may be used to receive input, such as when the display is a touchscreen. The management device 104 may further include input elements 125, such as a keyboard, button, knobs, or the like, for receiving input of the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128.

Other devices, like smartwatch 130, fitness monitor 132 and device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user 108, such as to request a change or pause in dosage, or to request, initiate, or confirm delivery of a bolus of medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data.

The functionality described herein for the exemplary embodiments may be under the control of or performed by the control application 116 of the medicament delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another wearable device 134.

In the closed loop mode, the control application 116, 120 determines the medicament delivery amount for the user 108 on an ongoing basis based on a feedback loop. For a medicament delivery device that uses insulin, for example, the aim of the closed loop mode is to have the user's glucose level at a target glucose level or within a target glucose range.

In some embodiments, the medicament delivery device 102 need not deliver one medicament alone. Instead, the medicament delivery device 102 may deliver a first medicament, such as insulin, for lowering glucose levels of the user 108 and also deliver a second medicament, such as glucagon, for raising glucose levels of the user 108. The medicament delivery device 102 may deliver a glucagon-like peptide (GLP)-1 receptor agonist medicament for lowering glucose or slowing gastric emptying, thereby delaying spikes in glucose after a meal. The medicament delivery device 102 may deliver a gastric inhibitory polypeptide (GIP) or a dual GIP-GLP receptor agonist. In other embodiments, the medicament delivery device 102 may deliver pramlintide, or other medicaments that may substitute for insulin. More generally, the medicament delivery device 102 may deliver a medicament for managing and/or affecting glucose levels of the user 108. In other embodiments, the medicament delivery device 102 may deliver concentrated insulin. In some embodiments, the medicament or medicament delivered by the medicament delivery device may be a coformulation of two or more of those medicaments identified above. In an exemplary embodiment, the medicament delivery device delivers insulin; accordingly, reference will be made throughout this application to insulin and an insulin delivery device, but one of ordinary skill in the art would understand that medicaments other than insulin can be delivered in lieu of or in addition to insulin.

Insulin deliveries to the user 108 may be bolus insulin deliveries or basal insulin deliveries. Bolus insulin deliveries tend to offset the expected rise in glucose level of the user 108 from ingesting a meal or for correcting a persistently elevated glucose level (i.e., one that is persistently higher than a target glucose level). Boluses tend to be one-time deliveries for offsetting a meal or for correcting a glucose level and tend to be larger than bolus insulin deliveries. Insulin boluses may be delivered manually by the user 108, such as via a syringe, or may, in some exemplary embodiments, be delivered by the medicament delivery device 102. Basal insulin doses tend to be smaller than insulin bolus doses and are delivered periodically, such as once each operational cycle of the control approach of the medicament delivery device 102 (e.g., every 5 minutes). The aim of the basal insulin deliveries is to keep the user's glucose level within a target range that is desirable using small ongoing insulin doses.

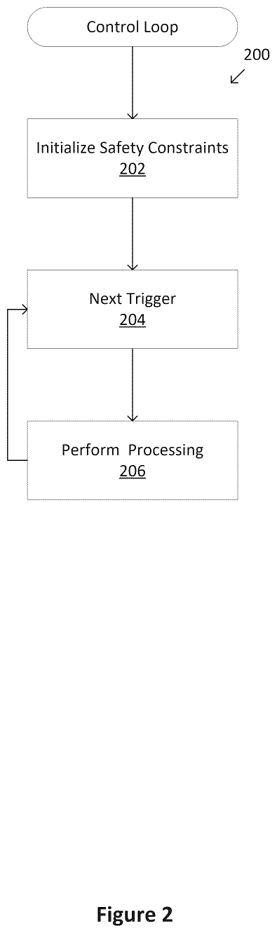

FIG. 2 depicts a flowchart of illustrative steps of a control loop that may be followed in exemplary embodiments in adjusting safety constraints of the medicament delivery system 100 to personalize the safety constraints based on recent activity. The steps may be performed by or initiated by control applications 116 or 120 or more generally by a control system. At 202, the safety constraints may be initialized. This may entail, for example, setting initial values or states for the safety constraints, or invoking default values, which may be based on population averages.

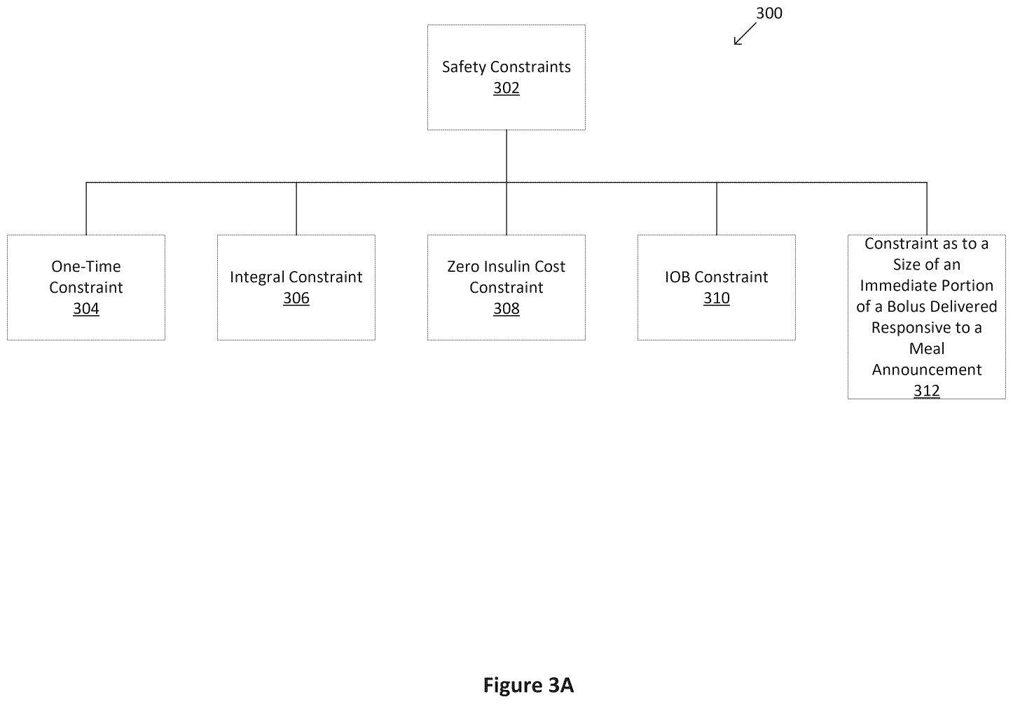

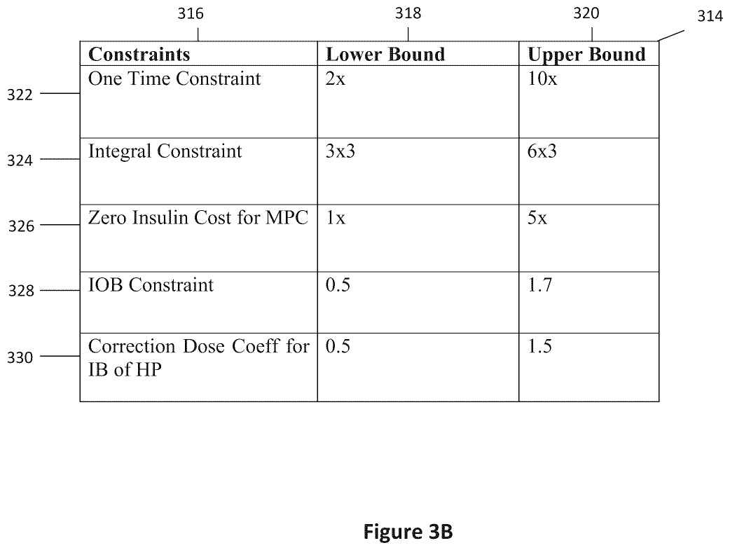

To better understand how the safety constraints are initialized, it is helpful to consider examples of safety constraints. FIG. 3A depicts a block diagram showing several illustrative varieties of safety constraints 302. A first variety of safety constraint may be a one-time constraint 304, which limits the dose of medicament that may be delivered by the medicament delivery device 102 in a single instance (such as per basal delivery dose). For example, where the medicament delivery device is an insulin pump, this constraint 304 would limit the dose of insulin that is permitted to be delivered as a single delivery or during a single cycle to a maximum amount.

A second variety of safety constraint may be an integral constraint 306. The integral constraint 306 may limit the cumulative dose of medicament that may be delivered over a time window. For example, the integral constraint 306 may limit the cumulative dose to X times the maximum per dose limit over-a time window (e.g., 3 hours, 12 hours, a day, etc.).

A third variety of safety constraint may be a zero insulin cost constraint 308. The zero cost insulin constraint 308 may refer to a coefficient that specifies what multiple of a baseline basal dose (such as a basal dose specified in a basal profile or a basal dose derived from a standard formula like one half of total daily insulin (TDI) divided by the number of cycle is a day) is subtracted from a candidate basal insulin dose to determine an insulin cost as part of an insulin cost component of a cost function. Insulin cost is zero for deviations that are not greater than the multiple of the baseline basal dose that is specified by this safety constraint. The insulin cost component is a portion of a cost function that is used in model predictive control (MPC) approaches to determine a next basal medicament dose for the user. For example, the cost may be:

Cost=Weighted Glucose Cost+Weighted Insulin Cost

The insulin cost reflects a penalty when the basal insulin dose differs positively from the baseline basal insulin dose. The glucose cost may reflect a penalty reflective of a positive deviation of a predicted glucose level of the user from a target glucose level of the user. A higher value for the coefficient of the zero insulin cost constraint increases the aggressiveness of the control system, whereas a lower value for the coefficient of the zero cost insulin constraint decreases the aggressiveness of the control system. Aggressiveness refers to how quickly the control system responds to elevated glucose levels, such as by delivering larger insulin or other medicament doses.

A fourth variety of safety constraint may be an insulin on board (IOB) constraint 310. The IOB constraint 310 may limit the size of an insulin dose based on an IOB value for the user, IOB representing an estimate of prior insulin that has been delivered but has not yet acted or been metabolized in the body. In particular, the required IOB for the user is adjusted to be the product of the current IOB required and a coefficient. The coefficient acts as a safety measure influencing the required IOB constraints. IOB constraints represent the variance between required IOB and total calculated IOB, with higher required IOB leading to stricter constraints. Increasing the coefficient for required IOB allows for more insulin delivery in cases where the system encounters the IOB constraint limit due to hyperglycemia. Conversely, in hypoglycemic scenarios, a conservative approach is necessary. Lowering the required IOB tightens the IOB constraint, providing enhanced protection as the system is less likely to exceed the IOB limit. By controlling the IOB, a system can optimize insulin delivery for both hyperglycemic and hypoglycemic conditions.

A fifth variety of safety constraint 312 may concern what portion of a bolus that is to be delivered responsive to a meal announcement entered by the user. For instance, the user may simply select a meal button to indicate that the user is about to consume a meal. The user does not specify a meal size or the number of carbohydrates in the meal. In response, the control system delivers a bolus of medicament of a given dose. This safety constraint specifies what portion of the bolus dose is delivered immediately. The portion to be delivered may be specified by a coefficient. The safety constraint 312 may limit the magnitude of the immediately delivered portion of the bolus to a specified percentage of the total bolus to be delivered. For instance, the constraint 312 may dictate that only 80% of the bolus can be delivered immediately, with the remaining portion of the bolus being delivered after a delay and perhaps delivery of the remaining portion may be conditioned on glucose levels of the user. The percentage delivered immediately may be specified by a constraint in the form of a coefficient (such as 0.8) of the total bolus dose amount.

It should be appreciated that this enumeration of the safety constraints is intended to be merely illustrative and not limiting. Other safety constraints may be used in some embodiments. Hence, fewer safety constraints may be used, more safety constraints may be used, or even a completely different set of safety constraints may be used in some exemplary embodiments.

As mentioned above, at 202, the values for the safety constraints may be initialized. For example, for the safety constraints depicted in FIG. 3A, the values may set as follows: the one-time constraint may be set at 4 times a baseline basal dose, the integral constraint may be set at 4 times the baseline basal dose over a three hour time window. The JOB safety constraint may be set so that the coefficient has a value of 1, the zero cost function constraint may be set at 2 times the baseline basal dose, and the-safety constraint that specifies a coefficient that sets what portion of a bolus responsive to a meal announcement is immediately delivered may be set at 0.8. It should be appreciated that the safety constraints may be initialized at other values than the examples listed above.

At 204, a next trigger occurs. The trigger may be a time period being reached, such as a next day, a next hour, or a next cycle being reached. The trigger may also be an event, such as the glucose level of the user being out of a desired range, surpassing a threshold, and/or the user experiencing a hyperglycemic event or hypoglycemic event. Once the trigger is reached, at 206, processing may be performed to adjust the safety constraints. The process may then repeat with step 204 waiting for the next trigger. As such, the safety constraints may be adjusted on an ongoing basis as warranted.

It should be appreciated that the values of the safety constraints may be bounded. FIG. 3B depicts a table 314 of safety constraints 316, exemplary lower bounds 381, and exemplary upper bounds 320. As shown in row 322, the one-time safety constraint may have a lower bound of 2 times the baseline basal dose and an upper bound of 2 times the baseline basal dose. Row 324 shows that the integral constraint lower bound may permit a maximum of 3 times the baseline basal dose per a 3 hour time window, and the integral constraint upper bound may limit the deliveries to 6 times the baseline basal dose over a three hour time window. Row 326 shows that the zero insulin cost constraint may have a lower bound of 1 times the baseline basal dose and an upper bound of 5 times the baseline basal dose. Row 328 shows that the JOB constraint coefficient may have a lower bound of 0.5 and an upper bound of 1.5. Row 330 shows that the safety constraint that specifies the coefficient that sets the percentage of the bolus dose that is delivered as an immediate bolus responsive to a meal announcement may have a lower bound of 0.5 and an upper bound of 1.5 for the coefficient value.

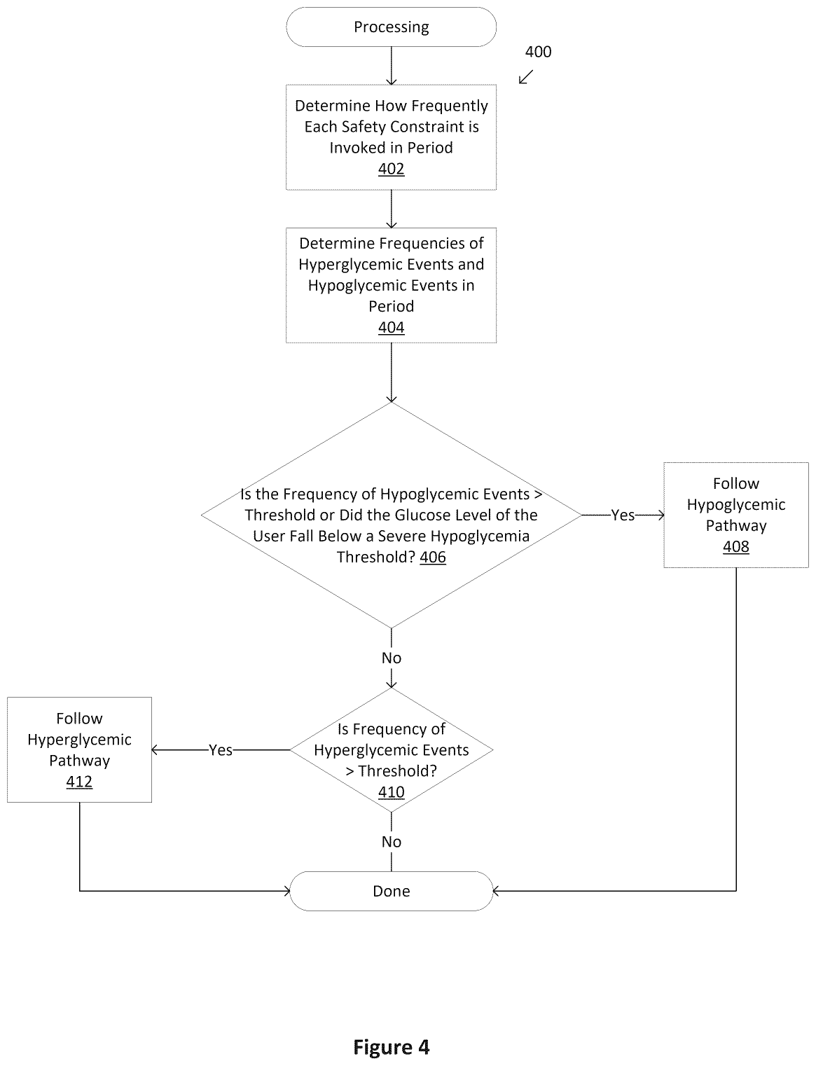

FIG. 4 depicts a flowchart 400 of illustrative steps that may be performed in exemplary embodiments to perform processing (see 202 in FIG. 2). At 402, it is determined how frequently each safety constraint is invoked in a period of interest to constrain doses delivered to the user 108. For instance, if the period of interest is a day, a determination is made of how often over the day each safety constraint is invoked to constrain a dose that is delivered. The period of interest could be several hours, a single hour, multiple operational cycles, or even a single operational cycle.

At 404, the frequencies of hyperglycemic events and hypoglycemic events that occur in the period of interest are determined. A hyperglycemic event may be, for instance, when the glucose level of the user is greater than a hyperglycemic threshold (e.g., greater than 180 mg/dL) during a cycle. A hypoglycemic event may be, for instance, when the glucose level of the user is less than a hypoglycemic threshold (e.g., less than 70 mg/dL) during a cycle. These thresholds may vary depending on factors such as whether a meal has recently been consumed and may be tailored to the user 108.

At 406, one or more criteria may be applied. Specifically, for example, a check may be made of whether the frequency of hypoglycemic events exceeds a threshold and a check may be made whether the glucose level of the user 108 fell below a severe hypoglycemia threshold. For example, the check may determine if the percentage of cycles with hypoglycemia events exceeds 1.5% of all cycles for a day, then at 408, the hypoglycemic pathway of processing may be followed. Similarly, if the glucose level of the user in the day fell below 55 mg/dL, then at 408, the hypoglycemic pathway of processing may be followed. Otherwise, a check at 410 is made whether the percentage of cycles having hyperglycemic events is greater than 35% for the day, then at 412, the hyperglycemic pathway of processing may be followed.

It should be appreciated that different thresholds may be used than the exemplary thresholds given above. Moreover, different criteria may be used to decide whether the hypoglycemic pathway or the hyperglycemic pathway are followed or not.

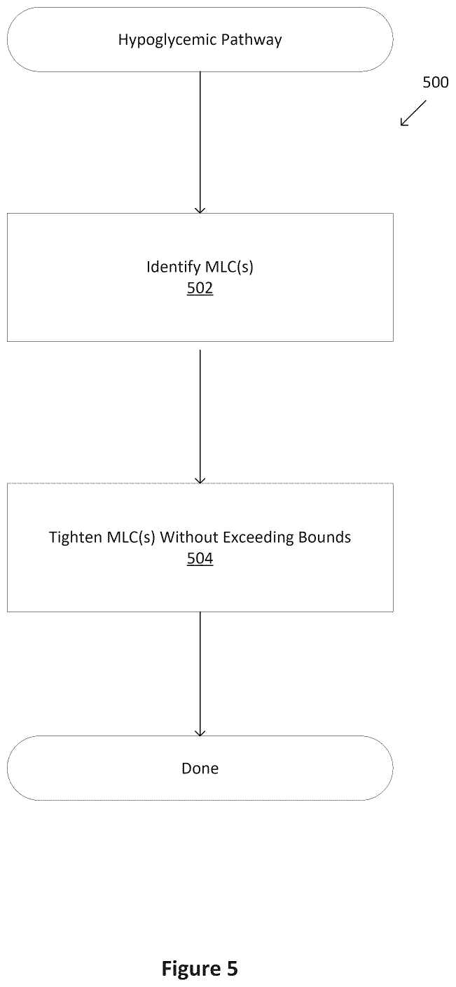

FIG. 5 depicts a flowchart 500 of illustrative steps that may be performed in exemplary embodiments as part of a hypoglycemic pathway. At 502, the most limiting constraint(s) (MLC(s)) is (arc) identified. The MLC may be the safety constraint that is invoked the most over the period of interest (e.g., a day) to constrain the dose of medicament delivered to the user by the medicament delivery device 102. In some embodiments, a single MLC is identified. In other exemplary embodiments, a set of multiple MLCs may be identified, such as the three safety constraints that were invoked the most. At 504, the MLC safety constraint(s) is (are) tightened to constrain medicament dose sizes and/or the rate at which glucose levels may be decreased via delivery of medicament without exceeding the bounds discussed above.

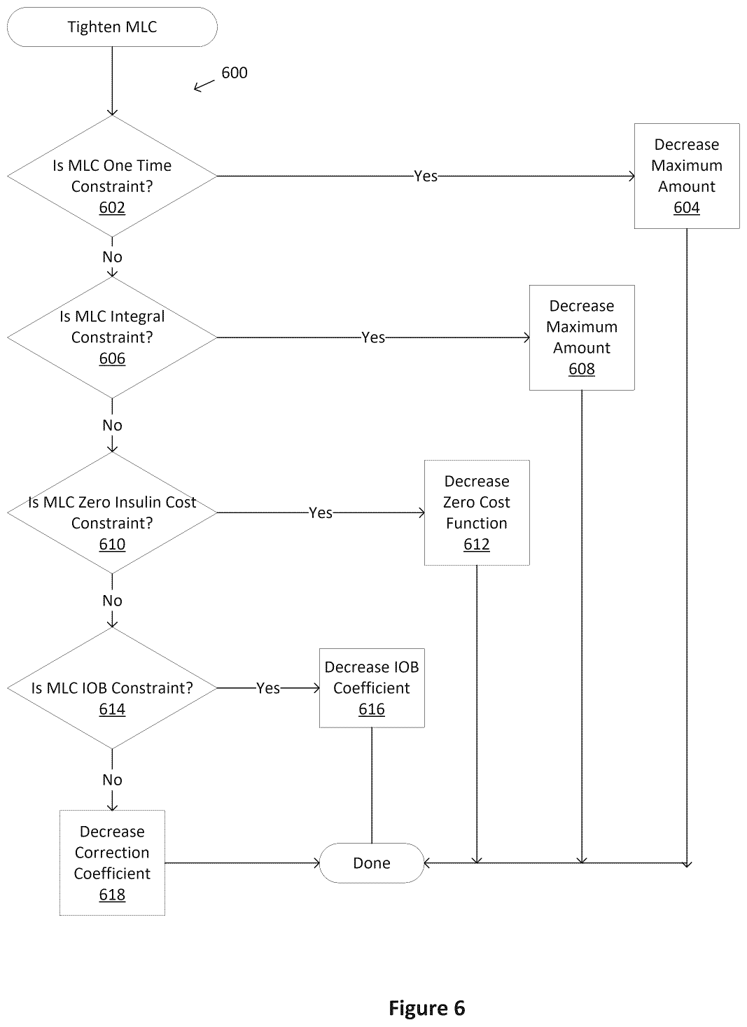

FIG. 6 depicts a flowchart 600 of illustrative steps that may be performed in exemplary embodiments to tighten an MLC. Where multiple MLCs are tightened, the process is iterated for each MLC. At 602, a check may be made of whether the “one-time constraint” is the MLC. If it is, at 604, the maximum one-time dose permitted by this particular safety constraint is decreased to a lower amount. For instance, the maximum may be decreased by 1 times, so, for example, the maximum dose could be decreased from 4 times the baseline basal dose to 3 times the baseline basal dose for a specified time window. If it is not, at 606, a check may be made of whether the “integral constraint” is the MLC. If the integral constraint is the MLC, at 608, the integral constraint may be tightened by decreasing the maximum doses permitted by the integral constraint. For example, the integral constraint may be reduced by 1. Thus, a maximum of 4 times the baseline basal dose over a 3 hour time window may be decreased to 3 times the baseline basal over the 3 hour time window. If the integral constraint is not the MLC, at 610, a check may be made of whether the zero insulin cost constraint is the MLC. If the zero insulin cost constraint is the MLC, at 612, the zero insulin cost constraint may be tightened by decreasing the constraint value. For example, the zero insulin cost constraint may be decreased by 1 times the baseline basal dose If the zero insulin cost safety constraint is not the MLC, at 614, a check may be made of whether the IOB constraint is the MLC safety constraint. If the IOB constraint is the MLC safety constraint, at 616, the JOB constraint may be tightened by decreasing the coefficient specified by this constraint. For example, the coefficient may be decreased by 0.1. If the IOB constraint is not the MLC, at 618, the coefficient that sets the percentage of the bolus dose that is delivered as an immediate bolus responsive to a meal announcement may be tightened by decreasing the coefficient. For example, the coefficient may be multiplied by 0.9 to yield an adjusted value. It should be appreciated that the modifications to the safety constraints may differ from the examples given.

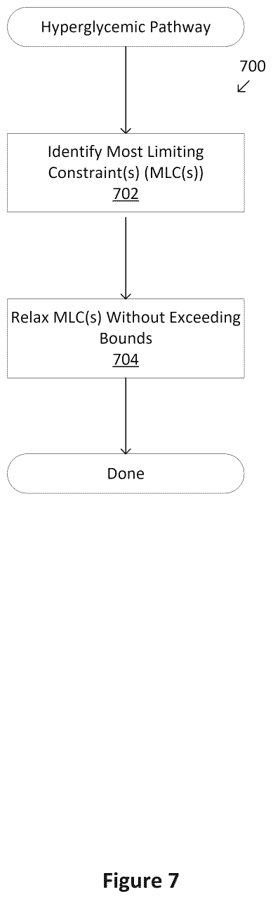

FIG. 7 depicts a flowchart 700 of illustrative steps that may be performed in exemplary embodiments in a hyperglycemic pathway. At 702, the MLC(s) may be identified for the period of interest. At 704, the MLC(S) may be relaxed without exceeding the bounds (see FIG. 3B). Such relaxing will cause an increase in the amount of medicament, such as insulin, that is delivered; and an increase will result in a reduction in the occurrence of hyperglycemia. The relaxing of the MLC(s) may help the control system to increase doses to more quickly counter hyperglycemia.

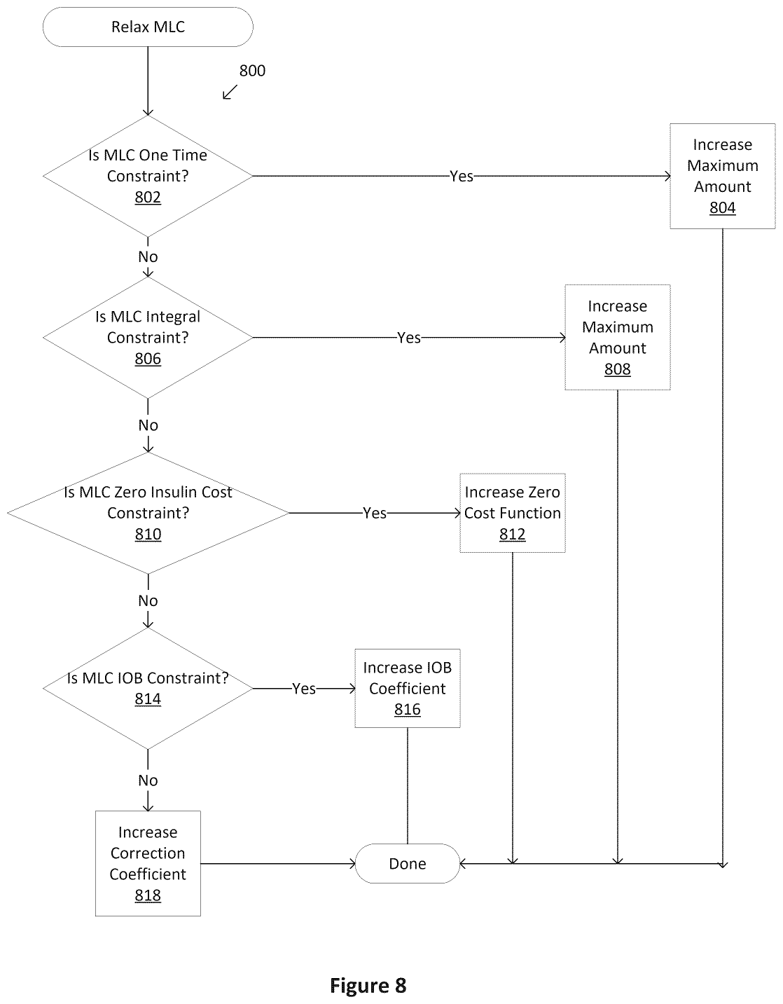

FIG. 8 depicts a flowchart 800 of illustrative steps that may be performed in exemplary embodiments to relax the MLC(s). The steps may be performed for each MLC if multiple MLCs are to be relaxed. At 802, a check may be made of whether the one-time constraint is the MLC. If the one-time constraint is the MLC, at 804, the one-time constraint may be relaxed by increasing the size of the one-time dose. For example, the constraint value may be increased by 1 times. If one-time constraint is not the MLC, at 806, a check may be made of whether the integral constraint is the MLC. If the integral constraint is the MLC, at 808, the integral constraint may be relaxed by increasing the dose size and/or frequency specified by the integral constraint. The dose size may be increased by 1 times, for example. If the integral constraint is not the MLC, at 810, a check may be made of whether the zero insulin cost constraint is the MLC. If the zero insulin cost constraint is the MLC, at 812, the zero insulin cost constraint may be relaxed, to increase the maximum glucose value of the user that is zero cost (i.e., the coefficient is increased to be a higher multiple of the baseline basal dose). The zero cost function may be increased by 1 times, for example. If the zero insulin cost is not the MLC, at 814, a check may be made of whether the IOB constraint is the MLC. If the IOB constraint is the MLC, at 816, the coefficient specified by the IOB constraint may be increased to relax the IOB constraint. For example, the IOB coefficient may be increased by 0.1. If the IOB constraint is not the MLC, at 818, the correction dose coefficient constraint may be relaxed by increasing the coefficient. For example, the coefficient may be multiplied by 1.1 to increase it by 10%.

While exemplary embodiments have been described herein, various changes in form and detail may be made without departing from the intended scope of the appended claims and equivalents thereof.

1. A medicament delivery system, comprising:
a non-transitory processor-readable storage media storing programming instructions;
a processor configured for executing the programming instructions to cause the processor to:
determine a frequency with which each of a plurality of safety constraints is encountered over a period from data for the period;
determine a number of hypoglycemic events and/or a number of hyperglycemic events that were experienced by a user over the period;
where the number of hypoglycemic events is determined, based on one or more criteria, determine whether the period should be categorized as a hypoglycemic period and where it is determined that the period should be categorized as a hypoglycemic period, tighten the selected one of the safety constraints that was encountered the most in the period; and
where the number of hyperglycemic events is determined, determine, based on one or more criteria, whether the period should be categorized as a hyperglycemic period and where it is determined that the period should be categorized as a hyperglycemic period, relax the selected one of the safety constraints that was encountered the most in the period.

2. The medicament delivery system of claim 1, wherein the programming instructions when executed by the processor further cause the processor to:
where it is determined that the period should not be categorized as a hypoglycemic period or a hyperglycemic period, determine that the period is a euglycemic period, not relaxing and not tightening the safety constraints.

3. The medicament delivery system of claim 1, wherein the one or more criteria used to determine that the period should be categorized as a hypoglycemic period include whether the determined number of hypoglycemic events for the period exceeds a threshold and whether the user experienced a glucose level in the period that was below a floor value.

4. The medicament delivery system of claim 1, wherein the plurality of the safety constraints includes a one-time constraint that places a maximum on an amount of medicament that can be delivered per a delivery, an integral constraint that places a maximum on an amount of medicament that can be delivered over multiple deliveries, a zero cost medicament constraint that specifies a penalization of an medicament cost term in a cost function for determining basal medicament doses, an insulin on board constraint.

5. The medicament delivery system of claim 1, wherein a hyperglycemic event is when a glucose level of the user exceeds a hyperglycemic threshold.

6. The medicament delivery system of claim 1, wherein the hypoglycemic events are when a glucose level of the user is below a hypoglycemic threshold.

7. A medicament delivery system, comprising:
a non-transitory processor-readable storage media storing programming instructions;
a processor configured for executing the programming instructions to cause the processor to:
analyze recent glucose levels of a user over an interval to determine whether glucose level control was satisfactory;
where glucose level control is determined to be unsatisfactory, adjust at least one of safety constraints imposed by a control system of the medicament delivery system, wherein the at least one safety constraint restricts dosing by the medicament delivery system to reduce a risk of hypoglycemia to the user; and
where glucose level control is determined to be satisfactory, not adjust the safety constraints.

8. The medicament delivery system of claim 7, wherein the analyzing the recent glucose levels of the user comprises determining a number of hypoglycemic events that occurred over the interval.

9. The medicament delivery system of claim 8, wherein the analyzing the recent glucose levels of the user comprises determining whether any of the recent glucose levels of the user over the interval was below a severe hypoglycemia glucose level threshold.

10. The medicament delivery system of claim 7, wherein the analyzing the recent glucose levels of the user comprises determining a number of hyperglycemic events that occurred over the interval.

11. The medicament delivery system of claim 7, wherein the adjusting adjusts a single one of the safety constraints.

12. The medicament delivery system of claim 7, wherein the adjusting adjusts multiple ones of the safety constraints.

13. The medicament delivery system of claim 7, wherein the programming instructions when executed by the processor further cause the processor to determine a frequency in which respective ones of the safety constraints were invoked to restrict dosing by the control system during the interval.

14. The medicament delivery system of claim 13, wherein the adjusting adjusts a most frequently invoked of the safety constraints during the interval.

15. The medicament delivery system of claim 13, wherein the adjusting adjusts multiple ones of more frequently invoked of the safety constraints during the interval.

16. The medicament delivery system of claim 7, wherein the adjusting adjusts one of the safety constraints that limits dosing to a maximum for a single medicament delivery or that limits dosing to a maximum over a period.

17. A non-transitory processor-readable storage medium storing computing program instructions that when executed by a processor cause the processor to:
analyze recent glucose levels of a user over an interval to determine whether glucose level control was satisfactory;
where glucose level control is determined to be unsatisfactory, adjust at least one of safety constraints imposed by a control system of the medicament delivery system, wherein the at least one safety constraint restricts dosing by the medicament delivery system to reduce a risk of hypoglycemia to the user; and
where glucose level control is determined to be satisfactory, not adjust the safety constraints.

18. The non-transitory processor-readable storage medium of claim 17, wherein the analyzing the recent glucose levels of the user comprises determining a number of hypoglycemic events that occurred over the interval.

19. The non-transitory processor-readable storage medium of claim 18, wherein the analyzing the recent glucose levels of the user comprises determining whether any of the recent glucose levels of the user over the interval was below a severe hypoglycemia glucose level threshold.

20. The non-transitory processor-readable storage medium of claim 17, wherein the analyzing the recent glucose levels of the user comprises determining a number of hyperglycemic events that occurred over the interval.
